(19) United States
(12) Patent Application Publication
(10) Pub. No.: US 2008/0306369 A1
(43) Pub. Date: Dec. 11, 2008

(54) DETACHABLE LATCHING MECHANISM

(75) Inventor: Shankar Murthy Udupa, Bangalore (IN)

Correspondence Address: PETER VOGEL;GE HEALTHCARE, 20225 WATER TOWER BLVD., MAIL STOP W492, BROOKFIELD, WI, 53045, US

(73) Assignee: GENERAL ELECTRIC COMPANY, Schenectady, NY (US)

(21) Appl. No.: 11/758,936

(22) Filed: Jun. 6, 2007

(51) Int. Cl. G05G 5/06; A61B 5/00

(52) U.S. Cl. 600/407; 74/527

(57) ABSTRACT

A detachable latching mechanism is disclosed herein. The latching mechanism for latching a first apparatus to a second apparatus comprises: a detachable housing configured to be attached to the first apparatus. The housing includes a first and second housing member forming a channel there between. The latching mechanism includes a first latch supported by the first housing member and biased to extend into the channel in a first direction and a second latch supported by the second housing member and biased to extend into the channel in a second direction opposite from the first direction. Further the latching mechanism is provided with a latching member having a first end configured to be attached to the second apparatus and a second end having a protrusion configured to push against the first and second latches to move them apart when the latching member is inserted into the channel. The movements of the latches are perpendicular to the movement of the latching member.

### FIELD OF THE INVENTION

This invention relates generally to latching mechanisms, and more particularly to, a detachable latching mechanism.

### BACKGROUND OF THE INVENTION

Latching mechanisms are well known for a variety of applications, including the latching of components or modules in machines. A latching mechanism is provided with a first part and a second part, the parts being engaged with each other for latching. One application for latching mechanisms is in medical imaging apparatus. Generally, in a medical imaging apparatus, a carriage assembly is used for carrying a mobile patient positioner to a gantry at the time of medical imaging. Examples of a medical imaging apparatus include a Magnetic Resonance Imaging (MRI) apparatus, a Computed Tomography (CT) scanner, a vascular imaging apparatus, etc. One example of a mobile patient positioner includes a patient table with a cradle. The patient table is configured for being docked to the imaging apparatus while imaging. Once the positioner is docked, the cradle is latched to the carriage assembly, which is taken to the gantry for imaging.

Generally a latch or hook mechanism is provided on the front section of the cradle and the carriage assembly to latch the cradle to the carriage assembly. In some example the cradle is provided with a rod and the carriage assembly will come and hold the rod to carry the cradle inside the gantry.

Typically, in a hospital or a clinical facility, it has been noticed that the cradle gets detached from the carriage assembly at higher patient weight. When the load on the positioner becomes high, the latching mechanism may fail to remain engaged. This may cause patient injury or damage to the cradle. Generally one part of the latch is attached to the carriage assembly, the other part is attached to the front section of the cradle. Hence in the event of damage to the latch section provided at the cradle, the whole cradle needs to be replaced. This is very cumbersome and expensive.

Thus there exist a need to provide a detachable latching mechanism that is capable of handling higher loads. There also exists a need to provide a detachable latching mechanism for use in medical imaging apparatus which is capable of handling the higher loads associated with heavier patients without becoming disengaged.

### SUMMARY OF THE INVENTION

The above-mentioned shortcomings, disadvantages and problems are addressed herein which will be understood by reading and understanding the following specification.

The present invention provides a latching mechanism for latching a first apparatus to a second apparatus, which may be used with medical imaging or other apparatus. The latching mechanism includes: a detachable housing configured to be attached to the first apparatus, the housing including first and second housing members forming a channel there between; a first latch supported by the first housing member and biased to extend into the channel in a first direction; a second latch supported by the second housing member and biased to extend into the channel in a second direction opposite from the first direction; and a latching member having a first end configured to be attached to the second apparatus and a second end having a protrusion configured to push against the first and second latches to move them apart when the latching member is inserted into the channel, the movements of the latches being perpendicular to the movement of the latching member.

In another embodiment, a positioner latching assembly is disclosed. The positioner latching assembly includes: a detachable housing having a plurality of housing members configured to define a cavity, the housing configured to attach to a support. A plurality of latches are provided on the housing members and are biased to extend into the cavity. Further a latching member is provided with one end configured to attach to a carriage assembly, the other end being provided with a protrusion configured to engage between the detachable housing and the plurality of latches, while latching. The latches are biased to move away from the cavity while latching, to allow the insertion of the latching member into the cavity and to re-extend back into the cavity after the latching member has been fully inserted into the cavity to prevent unlatching of the latching member.

In yet another embodiment, a medical imaging apparatus is disclosed. The medical imaging apparatus includes: at least one gantry configured with an imaging component; at least one carriage assembly configured with a patient support component; and a latching mechanism for latching the patient support component to the carriage assembly. The latching mechanism includes: (a) a detachable housing having a plurality of housing members configured to form a channel, the detachable housing fastened to the patient support component; (b) a first latching part associated with the carriage assembly, the first latching part including a latching member with a protrusion; (c) a second latching part provided on at least one of the housing members using a spring mechanism; the second latching part including a plurality of latches; wherein the protrusion of the latching member is configured to push against the latches to move them apart when the latching member is inserted into the channel, the movements of the latches being perpendicular to the movement of the latching member.

Various other features, objects, and advantages of the invention will be made apparent to those skilled in the art from the accompanying drawings and detailed description thereof.

### BRIEF DESCRIPTION OF THE DRAWINGS

FIG. 1 is a top view of a latching mechanism as described in an embodiment of the invention;

FIG. 2 is a top view of a latching assembly as described in an embodiment of the invention;

FIG. 3A is a top view of a latching assembly described with reference to FIG. 2, in a disengaged position;

FIG. 3B is a top view of a latching assembly described with reference to FIG. 2, in an engaged position;

FIG. 4 is a top view of a positioner latching assembly as described in an embodiment of the invention; and

FIG. 5 is a block diagram of a medical imaging apparatus using a latching mechanism as described in an embodiment of the invention.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

In various embodiments, a detachable latching mechanism is provided. The latching mechanism is configured to attach a first apparatus to a second apparatus. The latching mechanism is incorporated in a housing that is detachable from the device to which the latching mechanism is being fixed. This avoids the requirement of replacing any part of the device in the event of the failure of the latching mechanism. For example in medical imaging applications, the housing can be attached to a cradle and in the event of failure of latching mechanism, the latching mechanism can be replaced without the need to replace or alter any parts of the cradle.

In an embodiment the invention provides a positioner latching assembly. The positioner includes a carriage assembly and a support. The latching mechanism includes a detachable latching mechanism coupled to the support and includes at least two spring-loaded latches and a latching member with a protrusion. The latches along with the latching member are configured to secure the support with the carriage assembly.

In an embodiment the invention discloses a medical imaging apparatus with a detachable latching mechanism.

While the present technique is described herein with reference to medical imaging applications, it should be noted that the invention is not limited to this or any particular application or environment. Rather, the technique may be employed in a range of latching applications.

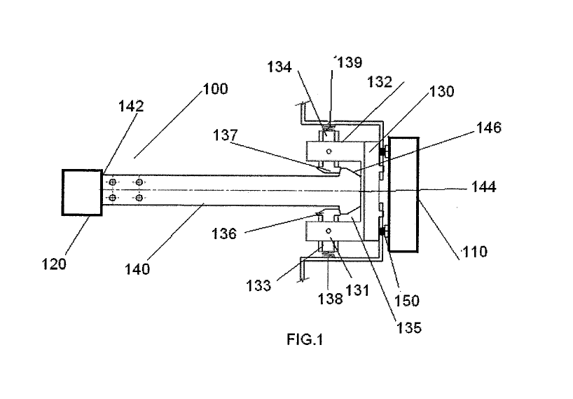

FIG. 1 is a top view of a latching mechanism as described in an embodiment of the invention. The latching mechanism 100 is configured to latch a first apparatus 110 with a second apparatus 120. The latching mechanism 100 is incorporated in a housing 130. The housing 130 is a detachable housing and in an embodiment it is attached to the first apparatus 110. The housing is configured to have a plurality of housing members. The housing members are configured to form a channel. In an embodiment shown, the housing 130 is provided with a first housing member 131 and a second housing member 132. The housing members together form a channel 135. A plurality of latches are provided on the housing members to facilitate latching. In an embodiment a first latch 133 and a second latch 134 are provided on the first housing member 131 and the second housing member 132, respectively. Each latch is provided with a head. The first latch 133 is provided with a first head 136 and the second latch 134 is provided with a second head 137. The latches are accommodated on the respective housing member in using a spring mechanism. In an embodiment, a first spring 138 is provided to accommodate the first latch 133 on the first housing member 131 and a second spring 139 is provided to accommodate the second latch 134 to the second housing member 132. A latching member 140 is provided for latching. The latching member 140 is provided with a first end 142 and a second end 144. The first end 142 is attached to the second apparatus 120 and the second end 144 is configured to have a protrusion 146.

The latches are provided extending into the channel 135. Initially the first latch 133 is extended into the channel 135 a first direction and the second latch 134 is extended into the channel 135 in a second direction. The first and second directions are opposite to each other. Basically the latches are provided facing each other. The spring mechanism including the first spring 138 and second spring 139 biases the first latch 133 and the second latch 134, respectively, to extend into the channel 135 and move away from the channel 135 while latching and re-extend into the channel 135 after the latching. In an embodiment each of the latches 133 and 134 is provided with a tapering head.

The latching member 140 provided in an embodiment is an elongated member with a protrusion 146 on one end. The latching member 140 is configured to move toward the housing 110 while latching. The second apparatus, which needs to be latched with the first apparatus, is attached to the first end of the latching member 142. The protrusion 146 on the second end of the latching member 144 is configured to push against the first and second latches 133, 134 to move them apart for inserting the latching member 140 into the channel 135. This will facilitate latching of the second apparatus 120 with the first apparatus 110. The springs 138, 139 associated with the latches 133, 134 will facilitate the movement of the latches away from the channel 135 when the latching member 140 is inserted into the channel 135 or when the protrusion 146 on the latching member 140 pushes the heads 136,137 of the latches, while the latching mechanism is being getting engaged. The springs 138, 139 also assist the latches in re-extending or returning to their positions in the channel 135 once the latching member 140 is completely inserted into the channel 135. The movements of the latches 133, 134 are perpendicular to the movement of the latching member 140.

For latching, the latching member 140 is inserted into the channel 135. While the latching member 140 is inserted, the first latch 133 and second latch 134 are being pushed away from the channel 135 in a direction opposite to each other. The configuration of the latches and the latching head is such that by applying minimal force, the latching member or the protrusion on the latching member will be able to push the latches apart and enter into the channel. In an example the latches can have a head with a plurality of tapering surfaces and the protrusion of the latching member can be an angular protrusion. The tapering head and the angular protrusion will allow the protrusion to enter into the channel with minimum pushing force. Once the protrusion is fully entered into the channel, the latches are allowed to return to their original location. Once the latching member enters into the channel completely, the force on the latches to stay away from the channel is reduced and the spring biases the latches to re-extend into the cavity. This will prevent unlatching of the latching member, as the protrusion of the latching member is now trapped or engaged between the housing and the latches.

In the embodiment shown in FIG. 1, the housing 130 is fixed to the first apparatus 110 using a fastener 150 such as a screw, bolt or other fastener. In the event of failure of the latching mechanism 100 or any components in the latching mechanism, the latching assembly can be removed from the latching by removing the fasteners 150. In other embodiments, the housing 130 is fixed to the first apparatus 110 by an adhesive, weld or similar means, though this will make it more difficult to remove the housing 130 from the first apparatus 110 should the latching mechanism 110 need to be removed.

In different embodiments there could be different structures for the housing member, channel, latches and or latching member. There could be more then one latch in a single housing member and all the latches could extend into the cavity and could be supported by the spring mechanism. The configuration of the latch head and /or the protrusion on the latching member can be varied. The structure, dimensions, angles, etc. can be varied based on the design and application.

In different embodiments the first apparatus and/ or the second apparatus may be configured to move each other in a mutually opposite direction for engaging.

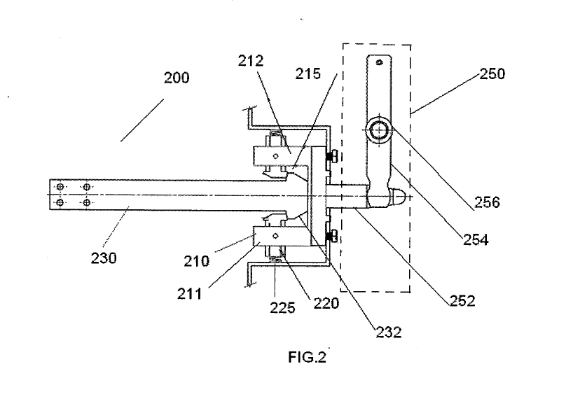

FIG. 2 is a top view of a latching assembly as described in an embodiment of the invention. The latching assembly includes a latching mechanism 200 and an unlatching mechanism 250. The latching mechanism 200 is incorporated in housing 210. The housing 210 is a detachable housing and incorporates a plurality of housing members 211, 212 configured to define a channel 215. Each housing member 211, 212 accommodates at least one latch 220 using a spring mechanism 225. The latches 220 are configured to extend into the channel 215. A latching member 230 is provided to facilitate latching. The latching member 230 used in an embodiment, is an elongated member with a protrusion 232 on one end. The latching member 230 is configured to move toward the housing 210 while latching. The protrusion 232 on the latching member is configured to push against the latches, so that they move away from the channel 215, when inserting the latching member 230 into the channel 215. The spring mechanism 225 associated with the latches 220 will facilitate the movement of the latches 220 away from the channel 215 while latching. The movements of the latches 220 are perpendicular to the movement of the latching member 230. The latches 220 re-extend into the channel 215 when the latching member 230 is completely inserted into the channel 215. This prevents unlatching of the latching member 230.

The latching assembly includes an unlatching mechanism 250. The unlatching mechanism 250 is configured to unlatch the latching mechanism 200 based on user requirement. The unlatching can be achieved in different ways. In an embodiment the unlatching mechanism 250 is provided with a release bar 252, which is accommodated in a gap (not shown) provided on the housing 210. At the time of unlatching, the release bar 252 is inserted through the gap in the housing 210 and a lever 254 associated with the release bar 252 is released so that the release bar 252 is pushed outwards. This will push the latching member 230 upward and this will automatically unlatch the latching mechanism 200. A cap 256 is provided to act as an assembly aid for locating the lever.

In an embodiment, where the latching mechanism is used in medical imaging applications, the latch can be disengaged upon moving the cradle downwards. The cradle moves down along with the table and the latch slides on the latching member. After traveling for some distance, the latch escapes from the latching member and enables unlatching.

In another embodiment unlatching of the latching assembly can be done by undocking the table. The latching member is pushed up from its location by the part of a pusher mechanism associated with the table enabling unlatching.

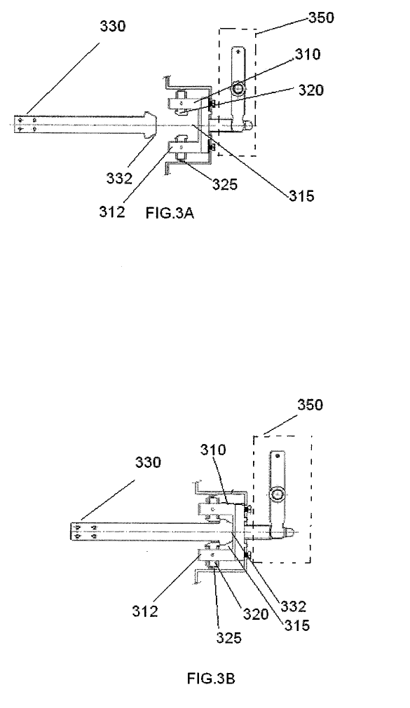

FIG. 3 is a top view of a latching assembly described with reference to FIG. 2, in a disengaged and engaged position respectively. FIG. 3A shows the latching assembly in a disengaged position. The latching assembly is provided with a latching mechanism and an unlatching mechanism 350. The latching mechanism is incorporated in housing 310. The housing 310 is a detachable housing and incorporates a plurality of housing members 312, configured to define a channel 315. Each housing member 312 accommodates at least one latch 320 using a spring mechanism 325. The latches 320 are configured to extend into the channel 315. A latching member 330 is provided to facilitate latching. The latching member 330 used in an embodiment, is an elongated member with a protrusion 332 on one end. The latching member 330 is configured to move toward the housing 310 while latching. The unlatching mechanism 350 is configured to unlatch the latching mechanism. FIG. 3B shows the latching assembly in an engaged position. The latching is achieved by pushing the latching member 320 to the channel 315 in the housing 310. The spring mechanism 325 associated with the latches 320 will facilitate the movement of the latches away from the channel 315 when the latching member 330 is inserted into the channel 315. In other words, the latches will move away from the channel 315, when the protrusion 332 pushes the latches 320 and this will facilitate the latching member to enter into the channel. Once the protrusion 332 is fully inserted into the channel 315, the spring mechanism 325 will assist the latches 320 in re-extending or returning to their original positions into the channel 315. The movements of the latches 320 are perpendicular to the movement of the latching member 330.

FIGS. 1-3 illustrate only exemplary constructional aspects of the latching assembly. However the construction of the latching mechanism can be varied based on different applications. The constructional and location aspects of the latching member, latches, spring mechanism, housing, number and constructional aspect of housing members etc can be varied based on desired applications.

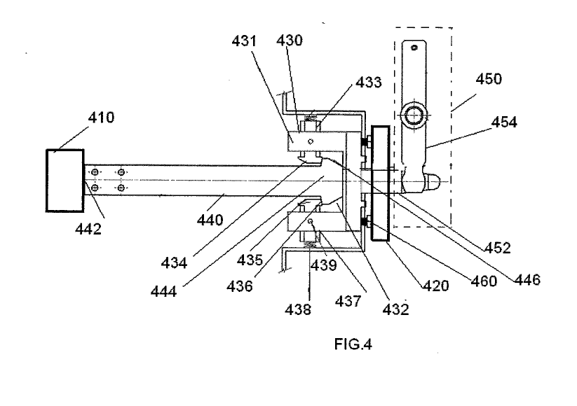

FIG. 4 is a schematic diagram of a positioner latching assembly as described in an embodiment of the invention. The positioner latching assembly is configured to latch a carriage assembly 410 with a support 420. The carriage assembly 410 is configured to carry or move the support 420. The support 420 may be configured to carry loads. The positioner latching assembly is incorporated in a housing 430. The housing 430 is a detachable housing and incorporates a plurality of housing members 431, configured to define a cavity 432. Each housing member 431 accommodates at least one latch 433 using a spring mechanism 438. Each latch 433 is provided with a head 434 that has a plurality of tapering surfaces. In an embodiment the latch 433 is provided with a first tapering surface 435 and a second tapering surface 436. The first tapering surface 435 being located in the direction of movement of a latching member 440, for latching and the second tapering surface 436 is located opposite to the first tapering surface 435. The tapering angle of the first tapering surface 435 is more than the tapering angle of the second tapering surface 436. The tapering configuration of the latches 433 facilitates easy latching with minimum push force and resistant to high pull force. However the design of the latch need not be limited to this and could vary based on the structure of the housing, latching member etc. The latches 433 are configured to extend into the cavity 432. The latching member 440 is provided to facilitate latching. The latching member 440 used in an embodiment, has a first end 442, configured to be attached to the carriage assembly 410 and a second end 444 provided with a protrusion 446. The latching member 440 is configured to move towards the housing 430 while latching. The protrusion 446 on the latching member is configured to push against the latches 433, so that they move away from the cavity 432, while inserting the latching member 440 into the cavity 432. The spring mechanism 438 associated with the latches 433 will facilitate the movement of the latches 433 away from the cavity 432 while latching. The movements of the latches 433 are perpendicular to the movement of the latching member 440. The latches 433 re-extend into the cavity 432 when the latching member 440 is completely inserted into the cavity 432. This prevents unlatching of the latching member 440.

As mentioned earlier the tapering angle of the first tapering surface 435 is more, compared to the second tapering surface 436, this will allow the protrusion 446 of the latching member to easily enter into the cavity 432 for latching. As and when the latching member 440 moves into the cavity 432, the spring mechanism 438 will bias the latches to move away from the cavity 432 in a direction perpendicular to the movement of the latching member 440. This will allow the latching member 440 to move inside the cavity 432 and to get the protrusion completely inserted into the cavity 432. As the tapering angle of the second tapering surface 436 is less in comparison with the first tapering surface 435, it helps in preventing the backward movement of the latching member 440, hence helps in avoiding unlatching.

The latches 433 are guided through the housing member 431 through a hole 437 or similar structure provided on the housing member 431. The guiding of the latches 433 through the holes 437 is made easy by the usage of the spring mechanism 438. The latches 433 are located in such a way that during latching, the latches 433 move in direction perpendicular to the direction of movement of the latching member 440 to facilitate latching. The tapering configuration of the latches 433 facilitates easy latching with minimum push force and resistant to high pull force. The varying stiffness of the spring mechanism 438 can accommodate different latch force.

The latches 433 can be accommodated to the housing member 431 using a fixing mechanism 439. The fixing mechanism can be a screw arrangement and this will control the stroke of the latches 433.

The positioner latching assembly is further provided with an unlatching mechanism 450. In an embodiment the unlatching mechanism 450 is provided with a release bar 452, which is accommodated in a gap( not shown) provided on the housing 430. At the time of unlatching, the release bar 452 is inserted through the gap in the housing and a lever 454 associated with the release bar 452 is released so that the release bar 452 is pushed outwards. This will push the latching member 440, more specifically the protrusion 446 upward and this will automatically unlatch the latching mechanism 430. A fastener 460 may be used to attach the housing 430 to the support 420. The fastener 460 is generally selected such that the latching mechanism can be detached from the support whenever required.

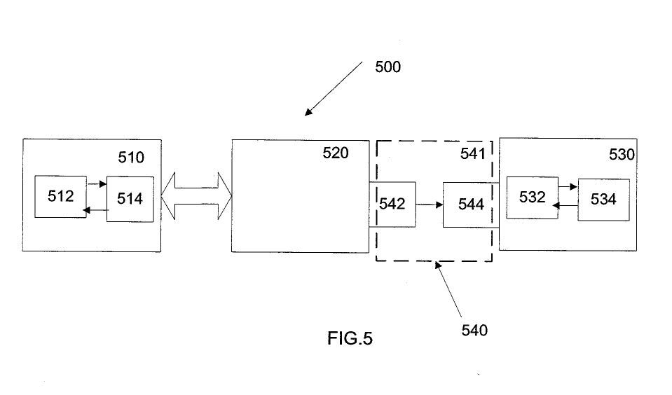

FIG. 5 is a block diagram of a medical imaging apparatus using a latching mechanism as described in an embodiment of the invention. The medical imaging apparatus 500 is provided with an imager 510, a carriage assembly 520 and a patient support component 530. A latching mechanism 540 is configured to latch the carriage assembly 520 with the patient support component 530. The imager 510 is provided with a gantry 512 having an imaging component 514. Examples of a medical imaging apparatus include a Magnetic Resonance Imaging (MRI) apparatus, a Computed Tomography (CT) scanner, a vascular imaging apparatus, etc. The patient support component 530 includes a trolley or a patient table configured for carrying a patient in substantially horizontal or tilted posture for medical imaging. In some instance the patient support component 530 is configured to be a cradle 532 attached to a patient table 534, the patient being positioned on the cradle 532. The latching mechanism 540 is provided with detachable housing 541. The latching mechanism 540 is attached to the front section of the cradle 532. The latching mechanism 540 is detachable from the patient support component 530, if required. The latching mechanism 540 is attached to the patient support component 530 using a fastener. The housing 541 is configured to have a plurality of housing members, configured to form a channel there between. The latching mechanism 540 further includes a first latching part 542 and a second latching part 544. The first latching part 542 is associated with the carriage assembly 520 and a second latching part is associated with housing 541. Generally the first latching part 542 includes an elongated latching member with a protrusion on one end, the other end being fixed to the carriage assembly 520. In some of the imaging applications the patient needs to be taken inside the gantry 512 and the cradle 532 carries the patient inside the gantry 512. The second latching part 544 is provided on the housing members and may include a plurality of latches loaded with a spring mechanism. In an embodiment the latches include a first latch provided on a first housing member and a second latch provided on a second housing member. The latches are accommodated to the housing member through a spring mechanism. The spring mechanism includes a first spring for associating the first latch to the first housing member and a second latch for associating the second latch to the second housing member. The first spring biases the first latch into the channel in the first direction and the second spring biases the second latch into the channel in the second direction. Various components of the latching mechanism are identical to the latching mechanism described in reference to FIGS. 1-3

During the imaging operation, the table 534 is docked to the imaging apparatus. The carriage assembly 520 having the first latching part 542 is moved towards the patient support component 530 for carrying the cradle 532 inside the gantry 512. The protrusion of the latching member in the first latching part 542 is moved towards the channel for latching. Generally the latching member will move towards the housing for latching and to carry the cradle to the gantry of the imaging apparatus. The protrusion is configured to push against the first and second latches to move them apart when the latching member is inserted into the channel. The movements of the latches are perpendicular to the movement of the latching member. Once the latching member is fully inserted into the channel, the first and second latches are biased to re-extend back into the channel, thereby latching the latching member in the channel to prevent unlatching.

In an example the material selected for latch and latching member are Teflon material and Delrin material respectively and this enables smooth unlatching while the table is moved down for unlatching the latch.

In an example for medical imaging applications, especially for magnetic resonance imaging application, the latching components can be made of non magnetic material so that it can be used in Magnetic Resonance Scanning.

Some of the advantages of the invention includes allowing the latching mechanism to be detachable from the device to which the latching mechanism is attached. The detachable latching mechanism can be replaced easily in the field without disturbing or changing any part of the device. Thus in the event of latching mechanism failure, the entire latching mechanism or components of the mechanism can be replaced without replacing any part of the main device to which the latching mechanism is attached. The latch member flexing is reduced to half in comparison with the existing solutions and hence the life of the latching mechanism is expected to be twice as longer. The position and size of the components in the latching mechanism is such that it can be used within a scan range in medical imaging application, without being scanned. The latching mechanism is simple and easy to manufacture and it reduces the latching failures. The latching mechanism is capable of handling a heavy load of about 500 lbs on the support. In medical imaging application, this is very helpful, as the patient support component has to carry different patients of different weights.

Thus various embodiments of the invention describe a latching mechanism housed in a detachable housing. Also in an embodiment positioner latching assembly and a medical imaging apparatus using the latching mechanism is disclosed. However the application of the latching mechanism need not be limited to positioners or medical imaging apparatus.

While the invention has been described with reference to preferred embodiments, those skilled in the art will appreciate that certain substitutions, alterations and omissions may be made to the embodiments without departing from the spirit of the invention. Accordingly, the foregoing description is meant to be exemplary only, and should not limit the scope of the invention as set forth in the following claims.

I claim:

1. A latching mechanism for latching a first apparatus to a second apparatus, comprising:
a detachable housing configured to be attached to the first apparatus, the housing including first and second housing members forming a channel there between;
a first latch supported by the first housing member and biased to extend into the channel in a first direction;
a second latch supported by the second housing member and biased to extend into the channel in a second direction opposite from the first direction; and
a latching member having a first end configured to be attached to the second apparatus and a second end having a protrusion configured to push against the first and second latches to move them apart when the latching member is inserted into the channel, the movements of the latches being perpendicular to the movement of the latching member.

2. A latching mechanism as in claim 1, wherein the first and second latches each has a head, and the protrusion of the latching member is configured to push against the heads of the first and second latches to overcome their respective biases to move them apart and allow the insertion of the latching member.

3. A latching mechanism as in claim 2, wherein the first and second latches are biased to re-extend back into the channel after the latching member has been fully inserted into the channel, thereby latching the latching member in the channel to prevent its removal.

4. A latching mechanism as in claim 1, further comprising a first spring and a second spring, wherein the first spring biases the first latch into the channel in the first direction and the second spring biases the second latch into the channel in the second direction.

5. A positioner latching assembly comprising:
a detachable housing having a plurality of housing members configured to define a cavity, the housing configured to attach to a support;
a plurality of latches supported by the housing members and biased to extend into the cavity; and
a latching member having a first end configured to attach to a carriage assembly and a second end having a protrusion configured to engage between the detachable housing and the plurality of latches, while latching;
wherein the latches are biased to move away from the cavity while latching to allow the insertion of the latching member into the cavity and to re-extend back into the cavity after the latching member has been fully inserted into the cavity.

6. A latching assembly as in claim 5, wherein each latch has a head, the heads of the latches being pushed apart from the cavity by inserting the protrusion into the cavity.

7. A latching assembly as in claim 6, wherein each head has a first tapering surface and a second tapering surface, the first tapering surface being located in the direction of movement of the latching member for latching and the second tapering surface is located opposite to the first tapering surface.

8. A latching assembly as in claim 7,wherein the first tapering surface is more tapered than the second tapering surface, preventing unlatching.

9. A latching assembly as in claim 5, further comprising a spring mechanism configured for biasing the latches to move away from the cavity, while the latching member being inserted into the cavity.

10. A latching assembly as in claim 9, wherein the spring mechanism is further configured to bias the latches to re-extend into the cavity, once the protrusion of the latching member being completely inserted into the cavity.

11. A latching assembly as in claim 10, wherein the spring mechanism comprises plurality of springs, the stiffness of the spring being based on latching force.

12. A latching assembly as in claim 5, wherein the movements of the latches being perpendicular to the movement of the latching member.

13. A latching assembly as in claim 5, wherein the housing is attached to the support using a fastener.

14. A latching assembly as in claim 9, further comprising an unlatch mechanism operable with the support.

15. A latching assembly as in claim 14, wherein the unlatch mechanism includes a release bar and a lever, the release bar being configured to lift the latching member.

16. A medical imaging apparatus, comprising:
at least one gantry configured with an imaging component;
at least one carriage assembly configured with a patient support component; and
a latching mechanism for latching the patient support component to the carriage assembly; said latching mechanism comprising:
(a) a detachable housing having a plurality of housing members configured to form a channel, the detachable housing fastened to the patient support component;
(b) a first latching part associated with the carriage assembly, the first latching part including a latching member with a protrusion;
(c) a second latching part provided on at least one of the housing members using a spring mechanism; the second latching part including a plurality of latches;
wherein the protrusion of the latching member is configured to push against the latches to move them apart when the latching member is inserted into the channel, the movements of the latches being perpendicular to the movement of the latching member.

17. A medical imaging apparatus as in claim 16, wherein housing members include a first and second housing member and the first latching part includes a first end and a second end, the first end being attached to the a carriage assembly and the second end has a protrusion.

18. A medical imaging apparatus as in claim 16, wherein the latches include a first latch and a second latch attached to the first member and the second member using a first spring and a second spring respectively.

19. A medical imaging apparatus as in claim 18, wherein the first and second latches each has a head, and the protrusion of the latching member is configured to push against the heads of the first and second latches to overcome their respective biases to move them apart and allow the insertion of the latching member.

20. A latching mechanism as in claim 19, wherein the first and second latches are biased to re-extend back into the channel after the latching member has been fully inserted into the channel, thereby latching the latching member in the channel to prevent its removal.
